(19) United States
(12) Patent Application Publication
(10) Pub. No.: US 2009/0118826 A1
(43) Pub. Date: May 7, 2009

(54) Valve prosthesis

(76) Inventor: Asghar Khaghani, Amersham (GB)

Correspondence Address: REINHART BOERNER VAN DEUREN S.C.;ATTN: LINDA KASULKE, DOCKET COORDINATOR, 1000 NORTH WATER STREET, SUITE 2100, MILWAUKEE, WI, 53202, US

(21) Appl. No.: 10/699,515

(22) Filed: Oct. 31, 2003

(30) Foreign Application Priority Data
May 1, 2001 (GB) 0110637.6
May 1, 2001 (GB) 0110639.2
May 1, 2002 (GB) PCT/GB02/01998

(51) Int. Cl. A61F 2/24; A61B 19/00

(52) U.S. Cl. 623/2.12; 623/2.1; 623/2.13; 128/898

(57) ABSTRACT

A heart valve prosthesis having a plurality of leaflets encircling a flow opening and of size to coapt to form a valve, each leaflet having a free outflow edge at the outflow end of the leaflet, wherein the free outflow edge forms a convex (relative to the leaflet) curve in the plane of the leaflet, and methods for forming such a valve. A stentless heart valve prosthesis suitable for replacement of the aortic root comprising an outer wall and a plurality of leaflets positioned inside the outer wall, encircling a flow opening and of size to coapt to form a valve, wherein the outer wall and leaflets are formed from material other than natural valve material, and methods of forming such a valve prosthesis.

### BACKGROUND OF THE INVENTION

### Field of the Invention

The present invention relates to a valve prosthesis, particularly a stentless valve bioprosthesis. The present invention also relates to a stentless valve prosthesis for aortic valve and root replacement, particularly a stentless composite valve prosthesis.

There are two principal classes of prosthetic subcoronary heart valve: the mechanical heart valve and the tissue heart valve. Mechanical heart valves are generally more durable but require patients to take anti-coagulants for the rest of their lives. Tissue heart valves in use are generally less durable; a high proportion may fail due to structural degeneration about seven years after implantation (depending on the age/activity of the recipient; see Turina et al (1993) Circulation 88(2), 775-781 for a review).

Tissue valves may be divided into stented and stentless valves. Stented valves are preferred for their relative ease of insertion, but are not as flexible as stentless valves. Stents also increase the outer diameter of the valve and therefore reduce the inner valve diameter for a given aortic size by half a gauge size.

Stentless tissue valves have great haemodynamic advantages over stented valves but unfortunately insertion of these valves in the sub-coronary position is technically challenging (usually requiring two suture rows) and, probably for that reason, is only performed by very few surgeons around the world.

Aortic valve and root replacement with a valved conduit is necessary when a patient's aortic wall is too weak/diseased to allow subcoronary valve replacement (i.e. replacement of the valve leaflets with retention of the aortic wall (valve root)). Root valves consist of a valve mechanism inside a tube which is intended to replace part of the aorta, including the aortic sinuses. In cases of severe aortic disease (aneurysm, dilated root, distorted root, small root and conditions such as Marfan's, replacement of the aortic root and part of the ascending aorta may become necessary.

Most currently available valves achieve this by inserting a stented valved conduit between the aortic annulus (outflow) and ascending aorta (composite valve). As mentioned above, the presence of a stent reduces the size of the effective valve area, thus compromising the left ventricular function.

Dacron™ has previously been used. However, untreated Dacron™ is too porous and results in massive blood leakage. More recently, gel-sealed Dacron™ has also been used. Unfortunately, the gel dissolves when brought into contact with fixation and preservation solutions such as glutaraldehyde or ethanol.

Studies in the past have suggested that bovine pericardium might develop calcifications and eventually dilate if used for root replacement.

Another currently available stentless valve substitute for root replacement consists of an actual porcine root. The arrangement of the coronary arteries on porcine roots is different from the human anatomy. As a result, modified surgical techniques such as rotating the valve or opening a new hole in the root for satisfactory implantation of the right coronary artery is required. The most important disadvantage is the lack of large size availability.

The muscular area of the porcine valve is not strong for sewing (after fixation) and needs to be covered by non-biological material to make it stronger for handling.

The length of porcine valves is not suitable for wider replacement of the ascending aorta and hence will require the addition of another interposition graft.

Another stentless root porcine bioprosthesis is an investigational stentless prosthesis with composite leaflets. Despite being stentless, the bioprosthesis is still bulky. The prosthesis is also available only in a limited range of sizes because of the limitations imposed by the size of available porcine valves (from which the prosthesis is constructed).

Bioprostheses, including stented and stentless valves, are reviewed in, for example, Cardiovascular Surgery Cardiac valvular replacement devices, residual problems and innovative investigative technologies Surgical Technology International VII, Jamieson & Lichtenstein and 25 Years of Heart Valve Replacements in the United Kingdom, A guide to types models and MRI safety, Maria-Benedicta Edwards, Heart Valve Registry, Hammersmith Hospital, 2000.

WO00/00107 describes a tissue aortic stentless valve constructed entirely of biological tissue, in which leaflets are attached to an adjacent leaflet at a commissure region, using an arrangement of tissue reinforcing commissure posts and a separate reinforcing rim strip.

WO01/05334 describes a flexible leaflet heart valve having leaflets with a scalloped and thickened free margin.

U.S. Pat. No. 5,713,953 describes a stentless valve prosthesis made from non-valve material, for example bovine pericardium, in which the outer sleeve and internal sheet which forms the valve flaps are each formed from an approximately rectangular or trapezoid sheet. The internal sheet is subjected to a shaping (distortion) process before assembly into the valve.

### SUMMARY OF THE INVENTION

We provide a heart valve prosthesis, which is considered to provide advantages in terms of durability and haemodynamic properties. In particular we provide a stentless tissue heart valve, which retains advantages of present stentless heart valves, and is considered to provide further advantages in terms of durability, haemodynamic properties and ease of surgical insertion.

We also provide a stentless heart valve prosthesis suitable for replacement of the aortic root and also part of the ascending aorta made from non-valve material, for example pericardium. The prosthesis may be made in a wide range of sizes. Coronary button holes may be made during surgery to suit the anatomy of the patient. In a preferred embodiment, the stentless annulus area of the valve is strong, scalloped and made especially for continuous suturing by extending the outer layer over the leaflets layer. The less bulky annulus enlarges the effective orifice area.

A first aspect of the invention provides a heart valve prosthesis having a plurality of leaflets encircling a flow opening and of size to coapt to form a valve, each leaflet having a free outflow edge at the outflow end of the leaflet, wherein the free outflow edge forms a convex (relative to the leaflet) curve in the plane of the leaflet.

A second aspect of the invention provides a stentless heart valve prosthesis suitable for replacement of the aortic or pulmonary root comprising an outer wall and a plurality of leaflets positioned inside the outer wall, encircling a flow opening and of size to coapt to form a valve, wherein the outer wall and leaflets are formed from material other than natural valve material.

Preferably, the leaflets and outer wall are formed from a sheet material or materials. More preferably, the outer wall is formed from a biological material (other than natural valve material), still more preferably a biological material covered or reinforced with a non-biological, biocompatible material. The non-biological material may protect the biological material against dilatation and/or calcification and may also assist in retaining the desired root shape, as discussed further below. Most preferably, the outer wall is formed from pericardium covered or reinforced on the outside with a woven fabric, preferably woven polyester (PET), for example Dacron™. The leaflets are preferably formed from a biological material, for example pericardium. The biological material leaflets are preferably without covering or reinforcement with a non-biological, biocompatible material. Thus, the outer wall may comprise a layer of biological material and a layer of a non-biological, biocompatible material.

Preferably, each leaflet in the second aspect of the invention has a free outflow edge at the outflow end of the leaflet, wherein the free outflow edge forms a convex (relative to the leaflet) curve in the plane of the leaflet.

The convex shape of the free edge of the leaflet in both the first and second aspects of the invention provides better coaptation than a straight or concave upper edge, as used in other tissue heart valves. The better coaption between the (preferably three) leaflets reduces the stress transmitted to the posts where they join and allows the valve to have a lower profile (i.e. height of the leaflets from the inflow edge to the outflow edge) than other valves, and also a larger coaptation area. A recent paper (Berk et al (2001) J Heart Valve Dis 10(1)) suggested that the main stresses working on bioprosthetic heart valves occur in the diastolic phase when the blood pushes back onto the top of the valve. The main stress is therefore exerted at the centre of the coaptation area rather than the posts. The convex shape of the free (outflow) edge of the leaflet provides a larger coaptation area allowing the leaflets to stabilise each other when the valve is closed, reducing the stress transmitted to the posts during the diastolic phase.

As will be apparent to those skilled in the art, the leaflets of the valve allow flow from the inflow to the outflow end of the valve when in the open position, but in the fully closed position the leaflets coapt to prevent flow back through the valve, i.e. from the outflow end to the inflow end.

A third aspect of the invention provides a method for forming a heart valve prosthesis comprising the step of forming a plurality of leaflets joined to encircle a flow passage and of a size to coapt to form a valve, wherein each leaflet has a free outflow edge at the outflow end of the leaflet, wherein the free outflow edge forms a convex (relative to the leaflet) curve in the plane of the leaflet.

A fourth aspect of the invention provides a method for forming a stentless heart valve prosthesis suitable for replacement of the aortic root comprising the steps of forming an outer wall and a plurality of leaflets positioned inside the outer wall, encircling a flow opening and of size to coapt to form a valve, wherein the outer wall and leaflets are formed from material other than natural valve material.

A fifth aspect of the invention provides a method for forming a heart valve prosthesis comprising the steps of: assembling the valve, by steps comprising forming a plurality of leaflets joined to encircle a flow passage and of a size to coapt to form a valve, and forming an outer sheet or wall joined to the leaflets around an inflow end and along commissures formed where adjacent leaflets join; after assembly of at least the leaflets and outer sheet or wall of the valve; shaping the leaflets and/or outer sheet or wall to a desired shape; fixing the leaflets and/or outer sheet or wall of the valve in the desired shape.

Preferably, the leaflets and/or outer sheet are shaped by inserting a shaping device into a pocket formed by a leaflet and the outer sheet or wall (sinuses of valsalva). The shaping device is preferably a ball (preferably substantially spherical) formed of a resilient material, for example cotton wool. The shaping device may have the effect of stretching portions of the leaflets and/or outer sheet into the desired shape.

Preferably, the leaflets are shaped (and then fixed) into a form that aids their coaptation.

Preferably, the outer sheet is also shaped to a desired shape and fixed in the desired shape.

Still more preferably, the shaping of the outer sheet (i.e. the change in shape, the stretch or deviation/distortion relative to the initial (unshaped) assembly) is in a portion of the outer sheet on the outflow side of the join between the outer sheet and the leaflets around the inflow end. Thus, the shaping is preferably in a portion of the outer sheet that corresponds to an aortic sinus in a natural aortic valve. Still more preferably, the shaping is such that the outer sheet has a conformation resembling that of a natural aortic sinus, for example has the appearance of a bulge when viewed from the exterior of the valve.

A sixth aspect of the invention provides a method for forming a stentless heart valve prosthesis comprising the steps of: forming a plurality of leaflets joined to encircle a flow passage and of a size to coapt to form a valve; forming an outer sheet or wall joined to the leaflets around an inflow end and along commissures formed where adjacent leaflets join; wherein the join between the outer sheet or wall and the leaflets around the inflow end is at the periphery of the leaflets, and the outer sheet or wall extends by a distance between 0.3 and 4 mm beyond the join with the leaflets at the inflow end, on the inflow side of the join, or (less preferably) the join between the outer sheet or wall and the leaflets around the inflow end is at the periphery of the outer sheet or wall, and the leaflet extends by a distance between 0.3 and 4 mm beyond the join with the outer sheet or wall at the inflow end, on the inflow side of the join.

It is preferred that the leaflets and/or outer sheet or wall (preferably both) are formed from material other than natural valve material. Still more preferably, the leaflets are formed from a sheet material, preferably a biological material, most preferably pericardium.

Preferably the extension is between 1 and 2 mm. The extension provides a sewing ring by means of which the valve prosthesis may be attached to the heart or vessel tissue.

It is preferred that the method of the sixth aspect of the invention further comprises the steps of after assembly of at least the leaflets and outer sheet or wall of the valve, shaping the leaflets and/or outer sheet or wall to a desired shape (preferably by inserting a shaping device into a pocket formed by a leaflet and the outer sheet or wall), and fixing the leaflets and/or outer sheet or wall of the valve in the desired shape. Preferences indicated in relation to the third to fifth aspects of the invention apply also to this aspect of the invention.

The valve prosthesis may be suitable for use as an aortic or pulmonary valve prosthesis, preferably an aortic valve prosthesis.

In relation to the third to sixth aspects of the invention, it is strongly preferred that each leaflet has a free outflow edge at the outflow end of the leaflet which forms a convex (relative to the leaflet) curve in the plane of the leaflet. This may be beneficial for the reasons indicated above in relation to the first and second aspects of the invention.

In relation to all aspects of the invention, it is preferred that the inflow edge of each leaflet is also convex (with respect to the leaflet) so that the valve has a shape (slightly scalloped) which complements the intended installation site, for example the subcoronary position or in the aorta, for example in place of part of the patient's aorta comprising the aortic sinuses, as known to those skilled in the art. Thus, it is preferred that the valve has three curved portions that complement the three aortic sinuses.

A preferred leaflet shape is shown in FIG. 1. Preferably, the leaflets form part of a single piece of material, for example pericardium, as discussed further below and shown in FIG. 1. Thus, the valve mechanism is preferably formed out of one flat piece of material, for example pericardium, such as bovine pericardium. The piece may have two or more (preferably three) distinct but connected leaflet regions, for example with convex inflow and outflow edges for each leaflet.

It is preferred that the curves of the inflow and outflow leaflet edges are smooth convex curves. For example, the inflow or outflow edge of each leaflet may have substantially the shape of a portion of the circumference of a circle. The radius of the circle may be approximately equal to the maximum height of the leaflet i.e. the maximum distance between the inflow and outflow edge of the leaflet. Thus, if h is the maximum height of the leaflet, the radius of curvature (r) of the inflow or outflow edge (which need not be the same) of each leaflet may be a×h, wherein a is between about 0.8 and 1.2, preferably between about 0.9 and 1.1, still more preferably between about 0.9 and 1.05, preferably about 1. The inflow or outflow edge may subtend an angle of between about 100° and 70° i.e. angle s or t in FIG. 1 may be between about 100° and 70°, still more preferably between about 90° and 80°. The width of the leaflet (i.e. from one commissure-forming region to the opposite commissure-forming region) may be b×h, wherein b is preferably between about 1.5 and 1, still more preferably between about 1.4 and 1.1, preferably between about 1.20 and 1.3, for example 1.24 to 1.27. Preferably, the commissure-forming edges are substantially parallel.

Preferably, the sum of the widths of the leaflets is π×the intended diameter of the valve. Thus, if the leaflets are formed from one piece of material, it is preferred that the width of the piece is π×the intended diameter of the valve. For a valve with three leaflets (for example), each leaflet width is preferably π×intended diameter/3.

The outer wall (when present) of the second, fourth, fifth and sixth inventions may be formed from one or more layers of sheet material. Preferably, it comprises (or is formed from) a sheet of biological material, preferably pericardium (outer sheet) and a sheet of reinforcing material, preferably untreated (i.e. non gel-sealed) woven polyester (PET), for example Dacron™, forming an outer protective layer.

The outer sheet for use in a heart valve prosthesis according, for example, to the first aspect of the present invention may preferably have a height (ho) that is slightly greater than the maximum height h of the leaflets, for example between about 0.3 and 24 or 14 mm, preferably between about 1 and 6 mm greater than h, still more preferably about 3 mm greater than h. The outer wall or sheet for use in a stentless heart valve prosthesis according to, for example, the second aspect of the present invention may preferably have a height (ho) that is between 1 and 15 cm, preferably between 4 and 12 cm, still more preferably between about 8 and 10 cm greater than the maximum height h of the leaflets.

The outer sheet in all aspects of the present invention preferably has a straight top (ie towards the outflow end of the valve) and a bottom edge (inflow end of the valve) that matches the shape of the leaflets. Thus, the bottom edge may have a scalloped appearance, having convex curved regions corresponding to the convex curved inflow edges of the leaflets. The outer sheet preferably has substantially the same (or slightly (for example 0.1 to 5, preferably 0.5 to 2 mm) greater) width as the total width of the leaflets (i.e. w in FIG. 1). Thus, wo in FIG. 2 (which shows an outer sheet for use with the inner (leaflet) sheet of FIG. 1) is preferably substantially the same as w in FIG. 1.

Exemplary leaflet dimensions and angles (which may be varied) are shown in FIG. 7.

The outer sheet is preferably formed from a single piece of material, but, much less preferably, may be formed from two or more pieces of material. The outer protective layer (when present) is preferably the same shape as the outer sheet and is likewise preferably formed from a single piece of material, or less preferably from two or more pieces of material. The outer sheet and outer protective layer are of sufficient height to be useful in replacing at least part of the aorta in a patient.

The height indicated above for the outer sheet for use in a heart valve according to, for example, the first aspect of the present invention is preferred because it facilitates assembly of the valve, for example by providing protection for the leaflets during assembly of the valve, and facilitates shaping of the valve by providing a pocket (in conjunction with a leaflet) into which a shaping device may be inserted, as described above. However, the outer sheet may have a lower height in the non-commissural regions, though this is not preferred. Preferably, the outer sheet is of sufficient height in the non-commissural regions to retain a shaping device sufficiently firmly to shape/stretch the leaflets as required.

The material from which the leaflets and outer sheet are formed is preferably pericardium, for example bovine pericardium, or kangaroo, porcine, ovine or equine pericardium. Still more preferably, the pericardium (or other tissue) is treated to reduce the risk of calcification, for example by treatment with ethanol (see, for example Vyavahare et al (1997) Circulation 95(2), 479-488). Ethanol treatment may result in reduction of lipid content and thereby reduce the risk of calcification. Pericardium is preferred as a result of its strength, flexibility and durability. Bovine pericardium may be preferred because of its durability, availability and size (which is greater than, for example, pig pericardium). Equine pericardium is also suitable. Porcine pericardium is also suitable. It is generally thinner than bovine pericardium and may be more suitable than bovine pericardium for smaller sized valves, for example valves intended for children.

Other suitable materials for forming valve prostheses or parts thereof according to the invention, for example according to the first aspect of the invention, may include other biocompatible materials, which may be biological material or synthetic, for example synthetic polymers. WO00/08107 and U.S. Pat. No. 5,713,953, for example, describe materials that may be suitable. However, pericardium is considered to be the most suitable material.

When biological tissue is used, the biological tissue is subjected to a cleaning operation after harvesting, as known to those skilled in the art. The tissue is examined and portions with suitable properties, for example, strength and thickness, and which are sufficiently homogeneous, are selected.

The selected tissue is then subjected to treatment operations intended to stabilise the elastic and mechanical strength of the tissue, and to render the tissue chemically inert with respect to blood. These fixation or stabilisation operations may be performed by immersing the tissue in solutions of glutaraldehydes with controlled pH. Treatment with anticalcifying agents, for example ethanol may be performed at the same time. The fixation operation generally involves the formation of stable cross links between the glutaraldehydes and the amino groups of the proteins constituting the tissue.

The treatment times may vary depending on the characteristics of the tissue to be fixed and the way in which the fixation is performed. The concentration of the fixative may be varied during the treatment process. For example, when glutaraldehyde is used, a prefixation phase may be performed with a solution of glutaraldehyde with a concentration of about 0.2%, which is increased in a final fixation phase to a concentration of about 0.5%.

In relation to aspects of the invention in which the leaflets and/or outer sheet are shaped after assembly of the leaflets and outer sheet, and are then fixed in the desired shape, it is preferred that the tissue forming the leaflets and/or outer sheet is not completely fixed. It is preferred that the tissue, for example pericardium, is partially fixed when assembled into the valve, and is subsequently subjected to a final fixation phase. The incompletely fixed tissue retains characteristics of plastic deformability which allow shaping operations to be performed thereon. The finally fixed tissue has different elastic characteristics such that, after a possible deformation (for example during operation of the valve, during which the leaflets move), the tissue tends to return spontaneously to the conformation assumed during fixation.

Thus, in aspects of the invention in which the leaflets and/or outer sheet are shaped after assembly of the leaflets and outer sheet, and are then fixed in the desired shape, it is preferred that the leaflets and outer sheet are assembled from pericardium which has not been fully fixed, and fixing the leaflets and/or outer sheet of the valve in the desired shape is performed by treatment with glutaraldehyde.

The outer protective layer (when present) is formed from a material that is biocompatible and resistant to fixation and preservation solutions such as glutaraldehyde or ethanol. It should be capable of assisting in retaining the root shape determined by the outer sheet and/or of protecting the outer sheet from calcification and/or rupture. The outer protective layer may be porous, so long as the outer sheet is not also porous. Thus, the outer protective layer may be woven polyester (PET), for example Dacron™ (or other suitable woven fabric) which has not been gel-sealed, particularly when the outer sheet is formed from pericardium (preferably bovine pericardium).

The joins between the leaflets and the outer sheet are preferably secured by means of sutures (stitching). The suture thread may be any suitable thread, as well known to those skilled in the art. Preferably, it is durable nylon or polyester thread. Suturing or stitching materials and techniques are described in the Examples, and also, for example, in WO00/59379 and U.S. Pat. No. 5,713,953. Moulding, gluing or welding may be used as an alternative (as mentioned in, for example, WO01/05334 or U.S. Pat. No. 5,713,953), but is less preferred.

In a preferred embodiment of, for example, the first aspect of the present invention, the valve may be assembled by stitching the leaflet sheet to the outer sheet, on the side which is to form the inside of the valve, for example as indicated in FIG. 3a and described in Example 1. The commissures may be formed by stitching through the outer sheet and the leaflet sheet. Reinforcement may be provided, for example in the form of strips of biocompatible material (for example a thin synthetic material, for example PTFE (for example Teflon™) through which the stitching passes, as discussed further in Example 1. The edges of the leaflet and outer sheets are abutted and stitched to complete the encirclement of the flow passage. The inflow edges of the leaflets are sewn onto the outer sheet (or, less preferably, vice versa). The leaflet sheet and outer sheet are preferably positioned so that the outer sheet extends by a distance between about 0.3 to 4 mm (preferably 1 to 2 mm) beyond the inflow edge of the leaflet. The join is preferably at the periphery of the leaflet sheet, so that the leaflet sheet does not extend beyond the join on the inflow side of the valve.

The reinforcing material is preferably confined to the commissural regions (for example may extend for no more than 5, 4, 3, 2 or 1 mm on either side of a commisure). It is preferred that the width of the reinforcing strip is 1 to 4 mm, preferably 2 mm. Preferably, it is of less than 3 mm or 2 mm thick in a plane perpendicular to the outer sheet; still more preferably, it is between 0.5 and 2 mm thick. It is preferred that the reinforcing material does not increase the overall diameter of the valve prosthesis (i.e. the valve prosthesis fits through the same sizing hole in a size gauging device with or without the reinforcing strips). The reinforcement may be woven polyester (PET), for example Dacron™ (DuPont).

In a preferred embodiment of, for example, the second aspect of the present invention, the valve may be assembled by stitching the leaflet sheet to the outer wall (outer sheet and outer protective layer), for example as indicated in FIG. 3b and described in Example 2. The commissures may be formed by stitching through the outer protective layer, outer sheet and the leaflet sheet. The abutted edges of the outer sheet/outer protective layer are sewn together above the level of the top (outflow) edge of the leaflets to complete the encirclement of the flow passage. The inflow edges of the leaflets are sewn onto the outer sheet (or, less preferably, vice versa) and outer protective layer. The stitching may pass through the outer protective layer and the outer sheet. The leaflet sheet and outer sheet are preferably positioned so that the outer sheet extends by a distance between about 0.3 to 4 mm (preferably 1 to 2 mm) beyond the inflow edge of the leaflet. The join is preferably at the periphery of the leaflet sheet, so that the leaflet sheet does not extend beyond the join on the inflow side of the valve.

Alternatively, but less preferably, the join between the outer sheet and the leaflets around the inflow end is at the periphery of the outer sheet, and the leaflets extend by a distance between 0.3 and 4 mm beyond the join with the outer sheet at the inflow end, on the inflow side of the join. In either arrangement, the extending material (preferably outer sheet or leaflet and outer protective layer when present) provides a sewing ring, by which the valve prosthesis may be secured in the desired position without having to sew through the leaflet and outer sheet on the outflow side of the leaflet-outer sheet join. It is preferred that the extending material is part of the outer sheet rather than the leaflet sheet, because this may minimise unwanted distortion of, or damage to, the leaflets on installing the valve prosthesis.

It is preferred that, when used according to, for example, the first aspect of the present invention, after assembly of the valve and, if appropriate, after shaping and fixing steps are carried out, the outer sheet on the outflow side of the join between the outer sheet and the leaflets is trimmed close to the join (on the outflow side of the join). Preferably, the outer sheet is trimmed to within 2 mm, still more preferably 1 or 0.5 mm of the join, particularly the join at the inflow end of the valve (as opposed to the joins in the commissural regions). More of the outer sheet (preferably about 1 to 2 mm) may be retained at the commissural regions, which may aid anchoring of the commissural regions at the site of insertion. The short trimming exposes the leaflets (FIG. 4) and renders the valve suitable for insertion in a subcoronary position. The trimmed valve allows preservation of the aortic root structures (i.e. minimises obliteration of the aortic sinuses) and hence preservation of the dynamic function of the aortic root structures. This may help in maintaining haemodynamic performance and maintaining low turbulence, and may thereby assist in prolonging the life of the prosthesis.

The short trimming of the outer layer, just above the attachment of leaflets, has the advantage that it may be installed using a single suture line, thus simplifying installation and thereby shortening the time required (when compared with prostheses requiring double suture lines). This technique also completely eliminates obliteration of the aortic sinuses, i.e. provides for total integrity of aortic sinuses.

When assembled from non-valve material, for example pericardium, the valves according to all aspects of the present invention may be made to any desired size. For example, the valve may be made to the following sizes: 17, 19, 21, 23, 25, 27 and 29 mm, which corresponds to the range of normal human adult aortic inner diameters. A size 15 valve may be made, which may be used in children. Larger valves may be made, for example for use in large animals, for example horses. Valves of sizes suitable for use in other experimental animals, for example sheep, may also be made.

A further aspect of the invention provides a valve obtained or obtainable by a method of the invention.

A further aspect of the invention provides a stentless heart valve prosthesis comprising a plurality of leaflets joined to encircle a flow passage and of a size to coapt to form a valve, an outer sheet joined to the leaflets around an inflow end and along commissures formed where adjacent leaflets join, wherein the join between the outer sheet and the leaflets around the inflow end is at the periphery of the leaflets, and the outer sheet extends by a distance between 0.3 and 4 mm beyond the join with the leaflets at the inflow end, on the inflow side of the join, or the join between the outer sheet and the leaflets around the inflow end is at the periphery of the outer sheet, and the leaflets extend by a distance between 0.3 and 4 mm beyond the join with the outer sheet at the inflow end, on the inflow side of the join.

In relation to this aspect of the invention, and the valve obtained or obtainable by a method according to the third and subsequent aspects of the invention, it is preferred that the leaflets are shaped/stretched to a desired shape and fixed in the desired shape. The outer sheet may also be shaped/stretched into a desired shape and fixed in the desired shape, still more preferably a shape resembling that of a natural aortic sinus, for example having the appearance of a bulge when viewed from the exterior of the valve.

It is preferred that the outer sheet is trimmed close to the join between the outer sheet and the leaflets on the outflow side of the join, as discussed above. This may be done prior to supply of the prosthesis to the user (surgeon) or may be done by the surgeon prior to installation of the valve prosthesis in the patient. Preferably it is done prior to supply to the user; this may result in more consistent trimming. This trimming may result in loss of any shaping of the outer sheet, as discussed above; thus, such shaping is of less importance if the outer sheet is to be trimmed close to the outer sheet/leaflet join.

A still further aspect of the invention provides a stentless heart valve prosthesis suitable for replacement of the aortic root comprising a plurality of leaflets joined to encircle a flow passage and of a size to coapt to form a valve, an outer wall joined to the leaflets around an inflow end and along commissures formed where adjacent leaflets join, wherein the outer wall and leaflets are formed from material other than natural valve material, wherein the join between the outer wall and the leaflets around the inflow end is at the periphery of the leaflets, and the outer wall extends by a distance between 0.3 and 4 mm beyond the join with the leaflets at the inflow end, on the inflow side of the join, or the join between the outer sheet and the leaflets around the inflow end is at the periphery of the outer sheet, and the leaflets extend by a distance between 0.3 and 4 mm beyond the join with the outer wall at the inflow end, on the inflow side of the join.

In relation to this aspect of the invention, it is preferred that the outer wall (outer sheet and outer protective layer) is shaped to a desired shape and fixed in the desired shape, still more preferably a shape resembling that of a natural aortic sinus, for example having the appearance of a bulge when viewed from the exterior of the valve.

The outer wall may be at the outflow end of the prosthesis. If the surgeon during the operation decides that a root replacement will be sufficient, the tubular extension can be cut down to the size of a root valve. For example, the tube (outer wall) can be cut just above the level of the leaflet outflow edges. Alternatively, the outer wall may be further trimmed so that the valve may be installed in the subcoronary position without replacement of any portion of the aorta, but this is not preferred.

In relation to all preceding aspects of the invention, it is preferred that the valve has three leaflets. Thus, for example, it is preferred that a sheet that forms the leaflets of the valve has regions providing three leaflets.

In relation to the first, third and fifth aspects of the invention, it is preferred that the valve is stentless. It is preferred that the valve comprises an outer sheet joined to the leaflets around an inflow end and along commissures formed where adjacent leaflets join.

It is preferred that the leaflets are formed from material other than natural valve material. Still more preferably, the leaflets are formed from a sheet material, as discussed above, yet more preferably a biological material, most preferably pericardium.

A further aspect of the invention provides a valve prosthesis according to the invention for use in medicine.

A further aspect of the invention provides the use of a valve prosthesis according to the invention in the manufacture of a medicament for the treatment of a patient in need of repair or replacement of a heart valve. Preferably, the heart valve in need of repair or replacement is the aortic valve. Optionally, the patient is in need of replacement of a portion of the aortic wall (i.e. a portion of the aortic root and optionally also ascending aorta).

The patient is preferably a human but may be a non-human animal, for example a horse.

A further aspect of the invention provides a method for repairing a heart valve comprising the step of providing a valve prosthesis of the invention, and suturing the valve prosthesis to the heart or blood vessel tissue of the patient. The method optionally includes the step of replacing the valve and part of the wall of the blood vessel with the valve prosthesis of, for example, the second aspect of the invention. The method may comprise the step of trimming the outer wall to the desired length, depending on the extent of aortic tissue to be replaced.

As will be apparent to those skilled in the art, the valve prosthesis is sutured in place after any necessary removal of the diseased heart valve leaflets, and any calcified material from the site of installation of the prosthesis.

A further aspect of the invention provides a method of repairing a heart valve comprising the steps of: (1) providing a valve prosthesis according to, for example, the first aspect of the invention, wherein the valve has an outer sheet; (2) if not already done, trimming the outer sheet close to the join between the outer sheet and the leaflets on the outflow side of the join; (3) suturing the valve prosthesis to the heart or blood vessel tissue of the patient with a single suture row. The suture is preferably continuous and the annulus of the valve is fortified with this technique in mind.

Preferably, the heart valve prosthesis is stentless.

Preferably, the outer sheet and/or leaflets (preferably both) is formed from material other than natural valve material. Preferences in relation to the trimming of the outer sheet are discussed in relation to earlier aspects of the invention. For example, it is preferred that the outer sheet is trimmed to within 0.5-1 mm of the outer sheet/leaflet join. The outer sheet may be marked with guidelines for trimming and/or stitching.

After annular suture the three commissures are fixed to the wall of the aorta incorporating the PTFE (for example Teflon™) strips using between 1-4 mm interrupted sutures for each commissure.

The prosthesis may be used or supplied with a holder, as is known for existing valve prostheses.

All documents referred to herein are, for the avoidance of doubt, hereby incorporated by reference.

The invention is now described in more detail by reference to the following, non-limiting, Figures and Examples.

### DESCRIPTION OF THE DRAWINGS

These and other advantages of the present invention are best understood with reference to the drawings, in which:

FIG. 1 is an example of a leaflet sheet providing three leaflets.

FIG. 2 is an example of an outer sheet suitable for use with the leaflet sheet of FIG. 1.

FIG. 3a is an assembly of the valve prosthesis according to, for example, the first aspect of the invention. The leaflet sheet is placed against the outer sheet. The outer sheet extends beyond the leaflet sheet at top and bottom. Reinforcing strips (1) (for example of a PTFE (for example Teflon™) material) are placed against the outer sheet on the opposite side to the leaflet sheet and aligned with the intended commissure regions. Double sutures (2) are made through the reinforcing strip, outer sheet and leaflet sheet. A single suture (5) is made along the periphery of the leaflet sheet to join the leaflet sheet to the outer sheet. The “seam allowance” (3) provides a sewing ring for securing the valve on implantation. The sewing ring may be reinforced or secured, for example with blanket stitch (4).

FIG. 3b is an assembly of the valve prosthesis according to, for example, the second aspect of the invention. The leaflet sheet is placed against the outer sheet. The outer sheet extends beyond the leaflet sheet at top and bottom. An outer protective sheet is placed against the outer sheet on the opposite side to the leaflet sheet. Double sutures (2) are made through the outer protective sheet, outer sheet and leaflet sheet. A single suture (5) is made along the periphery of the leaflet sheet to join the leaflet sheet to the outer sheet. The “seam allowance” (3) provides a sewing ring for securing the valve on implantation. The sewing ring may be reinforced or secured, for example with blanket stitch (4).

FIG. 4 is an assembled valve according to, for example, the first aspect of the invention after trimming, from a perspective (aortic) view and an outer view (laid open).

FIG. 5 is a view of an annulus of an assembled valve according to, for example, the first aspect of the invention (ventricular view). The leaflet/outer sheet join is visible, as is the reinforced sewing ring region and the reinforced commissures.

FIG. 6 is an assembled valve according to, for example, the second aspect of the invention. A. Side view and view from below, with detail of join. B. Inside view of valve.

FIG. 7 is a diagram and table showing exemplary dimensions and angles for valves of different sizes.

FIGS. 8a to 8d and echocardiogram results for Study ID KHA 03.

FIGS. 9a to 9c are echocardiogram results for Study ID KHA 10.

FIGS. 10a to 10e are echocardiogram results for Study ID KHA 11.

### DETAILED DESCRIPTION OF THE PREFERRED EMBODIMENT

### Example 1

### Subcoronary Stentless Valve Prosthesis

This example describes a subcoronary stentless valve prosthesis which is a preferred embodiment of aspects of the invention, in particular the first and third aspects.

The heart valve prosthesis is made from sheets of bovine pericardium, a very strong and durable tissue. The pericardium sheets are preserved in ethanol (20 to 80%) which has recently been shown to dissolve fat residues, thus reducing the risk of calcification. The suture thread is made of durable nylon or polyester thread.

### Valve Design

### The Leaflets

The valve mechanism is formed out of one flat piece of bovine pericardium. The sheet of pericardium is cut into three distinct but connected leaflets with convex upper (outflow) and lower (inflow) edges (FIG. 1). The convex outflow edges provide better coaptation than straight or concave outflow edges. This allows the valve to have a lower profile (i.e. height of leaflets) than a valve with straight or concave outflow edges. The shape of the leaflets also allows the leaflets to stabilise each other when the valve is closed, thus making them more resistant to the stress exerted during the diastolic phase and also resulting in less stress to the posts where the leaflets join.

### Outer Sheet

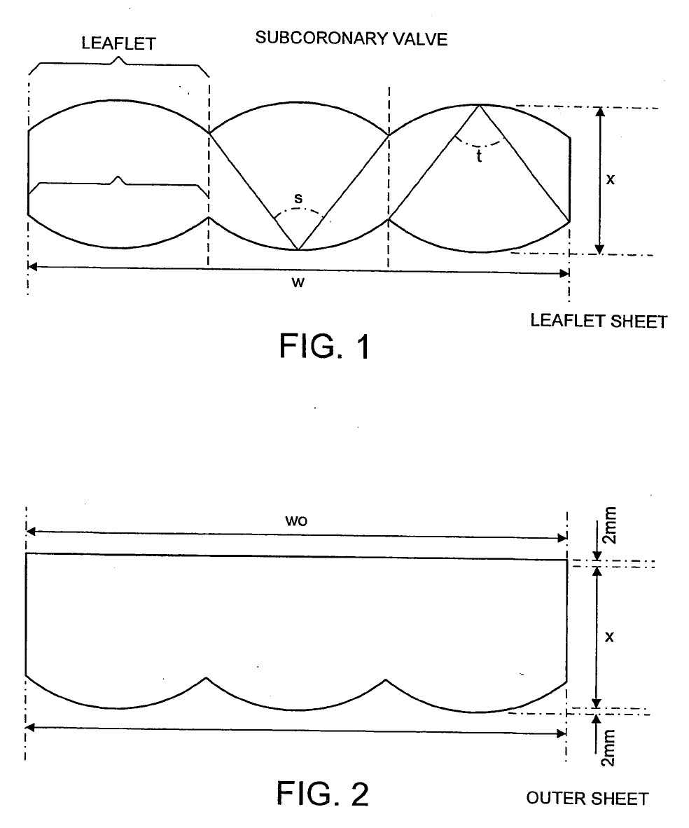

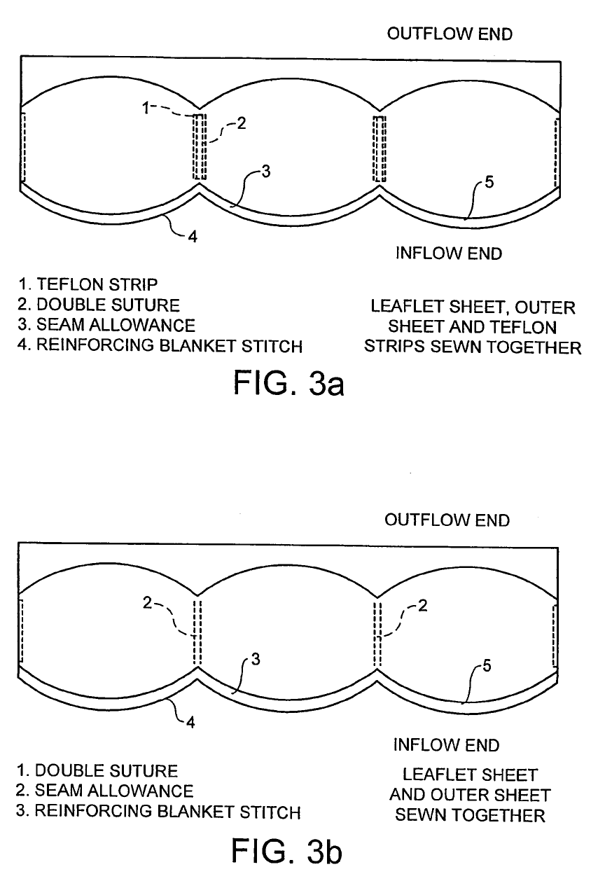

The sheet which forms the leaflets is sewn onto another, slightly higher flat sheet of bovine pericardium of the same width as the leaflet sheet (FIG. 2). This outer sheet has a straight top (outflow) edge, about 1-3 mm higher than the leaflet sheet. The bottom (inflow) edge of the outer sheet is about 1-2 mm longer than the leaflet sheet, i.e. extends about 1-2 mm beyond the inflow edge of the leaflet sheet.

### Valve Assembly

To reinforce the posts (which form the commissures) and facilitate their attachment to the inside of the aortic wall on implantation, small strips of reinforcing material, for example PTFE (for example Teflon™) are placed onto the outside of the outer sheet at the position of the posts. The PTFE strips are somewhat shorter towards the annulus (for example 1 to 5 mm shorter) than the length of the posts so as not to interfere with the overall diameter of the valve (the finished valve should still fit through the correct hole in a size gauging device, i.e. the size that it would fit through in the absence of the strips). The strips extend between 1-2 mm beyond the attachment of commissures. Leaflet sheet, outer sheet and PTFE strips are then sewn together by two rows of continuous stitching at each of the three commissures/posts, with the abut edges of the sheets forming the third post.

The scalloped bottom (inflow) edge of the leaflet sheet is then sewn onto the outer sheet using double sutured stitching, leaving a 1-2 mm “seam allowance” on the outer sheet. This seam allowance forms a sewing ring, but retains the original scalloped shape of the bottom edge. Sewing along this sewing area (when installing the valve), rather than through the leaflet and outer sheet also has the benefit of leaving the leaflets undamaged and without unwanted distortion. For reinforcement purposes and to keep the bottom edge of the outer sheet tidy the bottom edge is reinforced with suturing, for example using a blanket suture. The stitching may also provide a guide for the surgeon, which may help to avoid piercing the leaflets. The single layer of pericardium tissue is strong enough to fasten the valve to the aortic wall.

### Shaping and Fixation

Once the valve is assembled, cotton wool balls are packed tightly into the three pockets formed in the closed valve between the leaflets and the outer sheet (sinuses of valsalva). The cotton wool balls give the valve leaflets their characteristic shape and also cause three slight bulges in the outer sheet, mimicking the natural aortic sinuses. The valve is then fixed into this shape in glutaraldehyde.

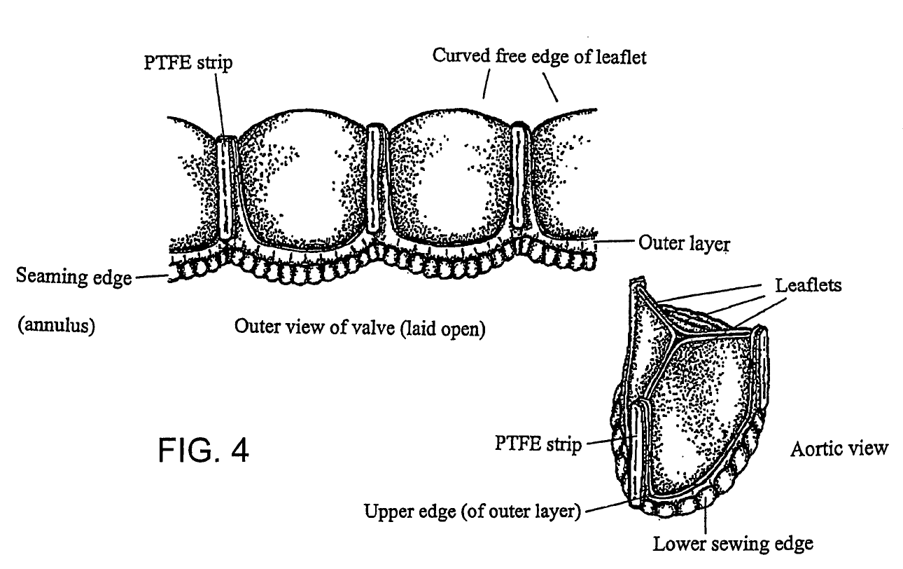

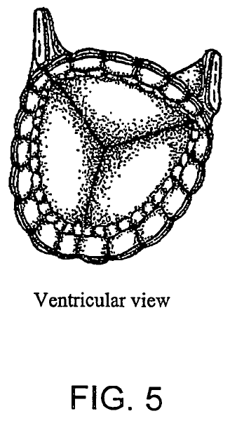

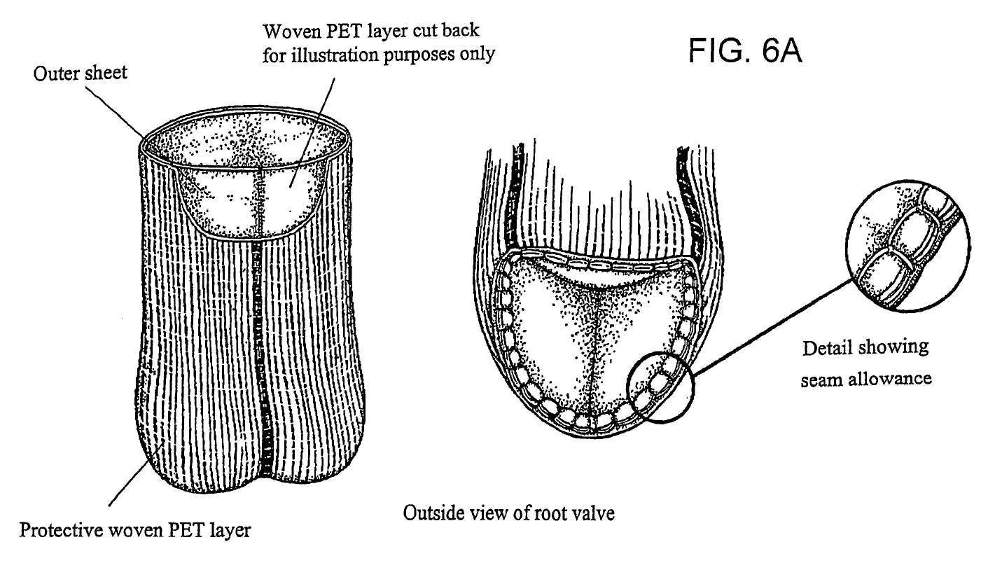

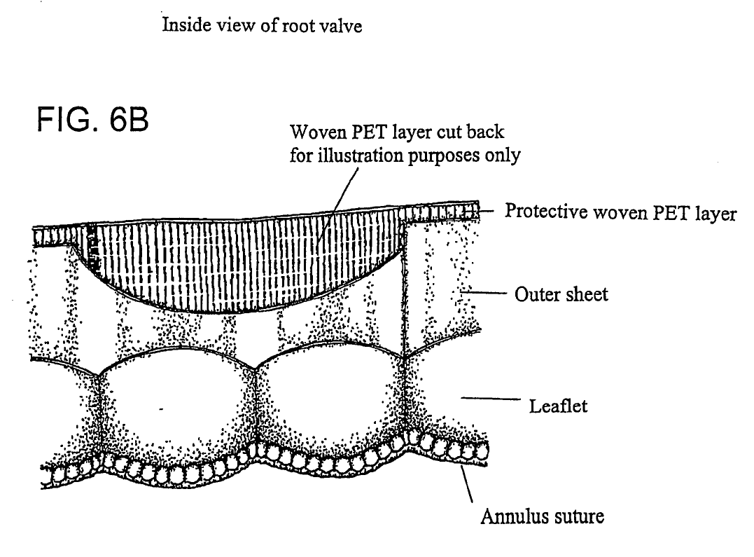

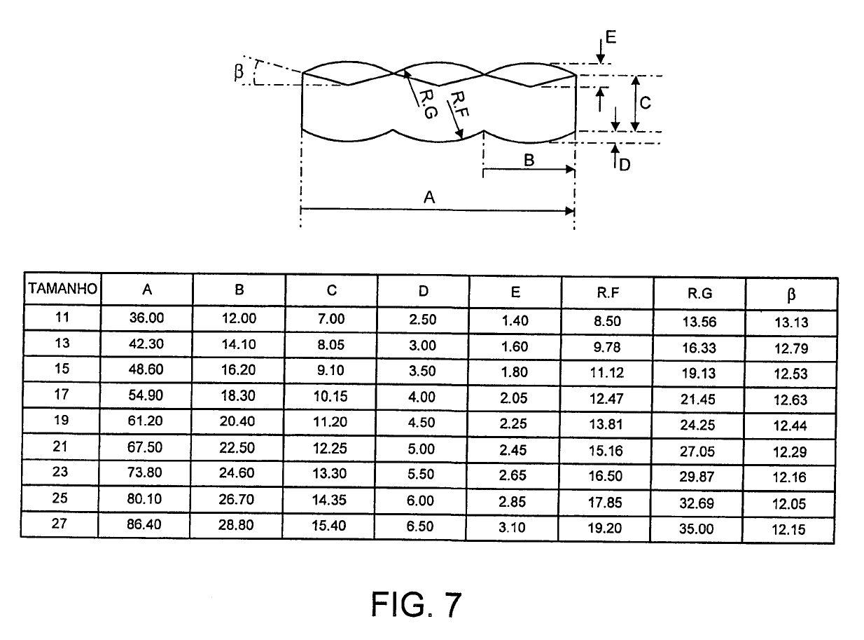

Finally, the outer sheet is cut closely around the sewing edges under a microscope, thus exposing the leaflets (FIG. 4).

The valve is fixed/preserved in a glutaraldehyde 0.4% buffered saline solution pH 4.7 and supplied sterile in a glutaraldehyde 0.2% solution.

### Using the Valve

This valve is designed for insertion into the subcoronary position following removal of a patient's diseased valve leaflets. Since all valve components are cut to size from flat sheets of pericardium, valves can be made to any size. The envisaged sizes are 17, 19, 21, 23, 25, 27 and 29 mm (considering the diameter at the sewing annulus area, at the inflow area) which corresponds to the range of normal human adult aortic inner diameters. A size 15 valve is also possible, which could be used in children.

### Example 2

### Root Replacement Stentless Valve Prosthesis

This example describes a root replacement stentless valve prosthesis which is a preferred embodiment of aspects of the invention, in particular the second and fourth aspects.

The leaflets and outer sheet of the heart valve prosthesis are made from sheets of bovine pericardium, a very strong and durable tissue. The pericardium sheets are preserved in ethanol (20-80%) which has recently been shown to dissolve fat residues, thus reducing the risk of calcification. The suture thread is made of durable nylon or polyester thread.

The outer protective sheet is formed from an untreated (i.e. non gel-sealed) woven polyester material (PET), for example Dacron™ (DuPont) in the form of a tube.

### Valve Design

### The Leaflets

### As Example 1.

### Outer Sheet

The outer sheet (onto which the leaflet sheet is sewn) is formed from a flat sheet of bovine pericardium of the same width as the leaflet sheet (FIG. 2). This outer sheet has a straight top (outflow) edge, about 8 to 10 cm higher than the leaflet sheet. The bottom (inflow) edge of the outer sheet scalloped in the same fashion as the bottom edge of the leaflet sheet, and is about 1-2 mm longer than the leaflet sheet, i.e. extends about 1-2 mm beyond the inflow edge of the leaflet sheet.

### Outer Protective Layer

The outer protective layer is made from a flat sheet of untreated woven polyester (PET), for example Dacron™ and is of substantially the same shape as the outer sheet.

### Valve Assembly

Leaflet sheet, outer sheet and outer protective layer are sewn together by two rows of continuous stitches at each of the three commissures, with the abutted edges of the sheets forming the third commissure. The two edges of the extended outer sheet are sewn together from the top of the third commissure upwards (i.e. towards the outflow end) using continuous double stitching.

The scalloped bottom (inflow) edge of the leaflet sheet is then sewn onto the outer sheet using double sutured stitching, leaving a 1-2 mm “seam allowance” on the outer sheet. This seam allowance forms a sewing ring, but retains the original scalloped shape of the bottom edge. Sewing along this sewing area (when installing the valve), rather than through the leaflet and outer sheet also has the benefit of leaving the leaflets undamaged and without unwanted distorted. For reinforcement purposes and to keep the bottom edge of the outer sheet tidy the bottom edge is reinforced with suturing, for example using blanket suture. The stitching may also provide a guide for the surgeon, which may help to avoid piercing the leaflets. The single layer of pericardium tissue is strong enough to fasten the valve to the aortic wall.

### Shaping and Fixation

Once the valve is assembled, cotton wool balls are packed tightly into the three pockets formed in the closed valve between the leaflets and the outer sheet (sinuses of valsalva). The cotton wool balls give the valve leaflets their characteristic shape and also cause three slight bulges in the outer sheet, mimicking the natural aortic sinuses. The valve is then fixed into this shape in glutaraldehyde.

A few continuous circular stitches in the sinuses of valsalva will keep outer and inner layers (pericardium and woven polyester (PET), for example Dacron™) together to facilitate the procedure for making button holes for coronary implantation.

The valve is fixed/preserved in a glutaraldehyde 0.4% buffered saline solution pH4.7 and supplied sterile in a glutaraldehyde 0.2% solution.

### Using the Valve

This valve has an extended outer tube of 8-10 cm for use as an ascending aorta replacement. If the surgeon during the operation decides that a root replacement will be sufficient, the tubular extension can be cut down to the size of a root valve. Since all valve components are cut to size from flat sheets of pericardium, valves can be made to any size. The envisaged sizes are 17, 19, 21, 23, 25, 27 and 29 mm (considering the diameter at the sewing annulus area, at the inflow area) which corresponds to the range of normal human adult aortic inner diameters. A size 15 valve is also possible, which could be used in children.

### Testing the Valves

The valves are tested to ensure that they are suitable for clinical use, for example that they conform to ISO 5840 standards.

Materials are tested as known to those skilled in the art. For example bovine pericardium may be tested for biocompatibility using the AMES test, and chromosome aberration, sensitization (Magnusson/Kligman), intracutane reactivity and haemocompatibility tests. Mechanical analysis may include stress-strain analysis and shrinkage temperature tests. The sutures used may be tested mechanically (tension stress) and for biocompatibility (cytotoxicity tests).

The valves may be subjected to mechanical and hydrodynamical testing according to standard protocols. For example, the valves may be tested over at least 200 million cycles. It may be evaluated in vivo, for example in juvenile sheep (see below). The sheep may be assessed for survival at 150 days and by blood analysis, echocardiogram analysis, as well as angioplasty, necroscopy and pathology.

Experimental Results.

A biological stentless aortic valve was implanted in the orthotopic position with coronary implantation in juvenile sheep. The animal model was selected for the following reasons: (1) The sheep model is an accepted and established method to evaluate the safety and preliminary efficacy of new or modified cardiac devices (ISO & FDA). (2) The size of sheep and the cardiac anatomy is comparable to humans and allows implantation of clinical size devices using standard surgical techniques.

Twenty four stentless pericardium valves were implanted using 17-19 mm sizes of Imperial Pericardium Valves. Thirteen animals had surgical details or early deaths (maximum of 3 days survival). Animals identified as KHA-01 to 11 were considered long term survival—more than 6 days and maximum of 176 days.

All animals underwent clinical examination before surgery. Rectal temperature, mucous membrane, skin integrity, lynphonodi, lungs and heart auscultation, abdomen palpation, behavior, reflexes and vital functions were checked. All animals were considered healthy. All animals received anthelmintic drug and antclostridial vaccine obeying to a sanity calendar for this specie. All animals received prophylactic antibiotics prior the surgery.

### Results

Study ID KHA 01: All animals underwent clinical examination before surgery. Rectal temperature, mucous membrane, skin integrity, lynphonodi, lungs and heart auscultation, abdomen palpation, behavior, reflexes and vital functions were checked. All animals were considered healthy. All animals received anthelmintic drug and antclostridial vaccine obeying to a sanity calendar for this specie. All animals received prophylactic antibiotics prior the surgery.

Study ID KHA 02: A 5-month-old, 32 kg female sheep (ear tag number 1033BR) underwent stentless aortic valve replacement. A bioprosthesis device (size 17 mm) was implanted with no complications. The surgical procedure and anesthesia recovery was uneventful and throughout the long-term holding period, the animal remained health and blood sampling was obtained per protocol. The animal was found dead on the 50^{th} post-operative day.

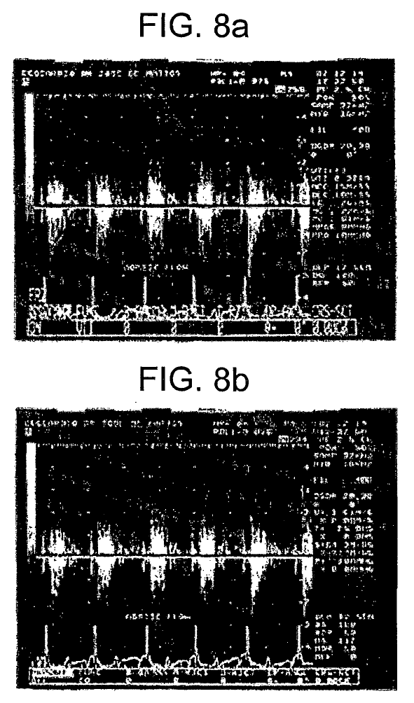

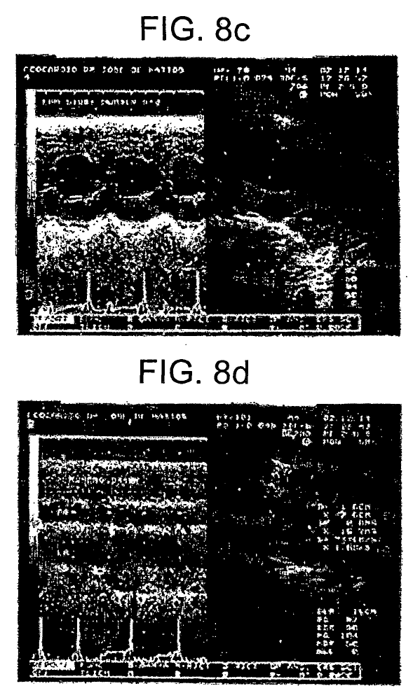

Study ID KHA 03: A 4-month-old, 35 kg neutered male sheep (ear tag number 940BR) underwent stentless aortic valve replacement. A bioprosthesis device (size 17 mm) was implanted with no complications. The surgical procedure and anesthesia recovery was uneventful and throughout the long-term holding period, the animal remained healthy and blood sampling was obtained per protocol. An angiogram was performed on the 169^{th} post-operative day. The animal was heparanized and sacrificed and underwent a necropsy. FIGS. 8a-d show the results of echocardiography analysis for this study. The aortic valve shows a normal Echo aspect with no evidence of anatomical or functional (dysfunctional) alterations.

Study ID KHA 04: A 4-month-old, 35 kg neutered male sheep (ear tag number 943BR) underwent stentless aortic valve replacement. A bioprosthesis device (size 19 mm) was implanted with no complications. The surgical procedure and anesthesia recovery was uneventful and throughout the long-term holding period, the animal remained health and blood sampling was obtained per protocol. The animal was found dead on the 50^{th} post-operative day.

Study ID KHA 05: A 4-month-old, 30 kg neutered male sheep (ear tag number 957BR) underwent stentless aortic valve replacement. A bioprosthesis device (size 19 mm) was implanted with no complications. The surgical procedure and anesthesia recovery was uneventful and throughout the long-term holding period, the animal remained healthy and blood sampling was obtained per protocol. The animal was found dead on the 8^{th} post-operative day.

Study ID KHA 06: A 4-month-old, 30 kg neutered male sheep (ear tag number 958BR) underwent stentless aortic valve replacement. A bioprosthesis device (size 17 mm) was implanted with no complications. The surgical procedure and anesthesia recovery was uneventful and throughout the long-term holding period, the animal remained healthy and blood sampling was obtained per protocol. The animal was found dead on the 6^{th} post-operative day.

Study ID KHA 07: A 4-month-old, 30 kg neutered male sheep (ear tag number 890BR) underwent stentless aortic valve replacement. A bioprosthesis device (size 19 mm) was implanted with no complications. The surgical procedure and anesthesia recovery was uneventful and throughout the long-term holding period, the animal remained healthy and blood sampling was obtained per protocol. The animal was found dead on the 13^{th} post-operative day.

Study ID KHA 08: A 5-month-old, 25 kg female sheep (ear tag number 948BR) underwent stentless aortic valve replacement. A bioprosthesis device (size 19 mm) was implanted with no complications. The surgical procedure and anesthesia recovery was uneventful and throughout the long-term holding period, the animal remained healthy and blood sampling was obtained per protocol. The animal was found dead on the 89^{th} post-operative day.

Study ID KHA 09: A 4-month-old, 25 kg female sheep (ear tag number 1165BR) underwent stentless aortic valve replacement. A bioprosthesis device (size 19 mm) was implanted with no complications. The surgical procedure and anesthesia recovery was uneventful and throughout the long-term holding period, the animal remained healthy and blood sampling was obtained per protocol. The animal was found dead on the 71^{st} post-operative day.

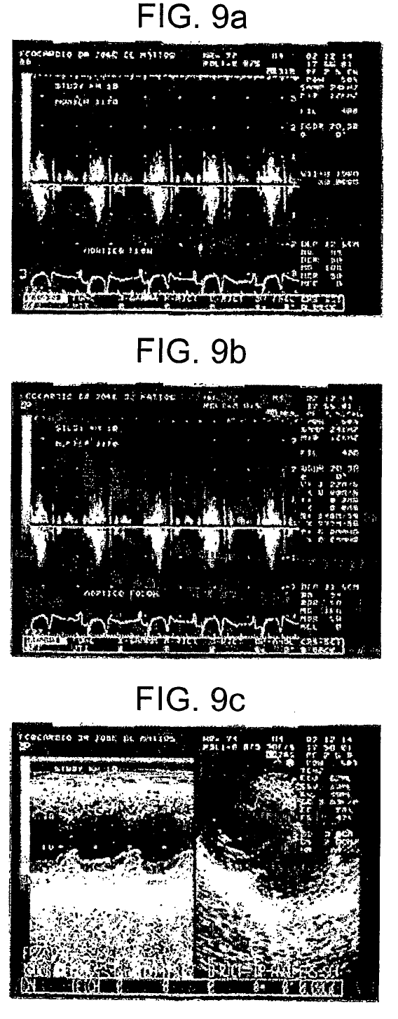

Study ID KHA 10: A 4-month-old, 27 kg neutered male sheep (ear tag number 1170BR) underwent stentless aortic valve replacement. A bioprosthesis device (size 19 mm) was implanted with no complications. The surgical procedure and anesthesia recovery was uneventful and throughout the long-term holding period, the animal remained healthy and blood sampling was obtained per protocol. An angiogram was performed on the 176^{th} post-operative day. A 4.95 L/min cardiac output was recorded without transvalvular pressure gradient. The animal was heparanized and sacrificed and recorded a necropsy. FIGS. 9a to 9c show the results of echocardiography analysis for this study. Leaflets of the aortic valve show mild alteration, the left coronary cusp presents some degree of restriction of the movement without stenosis.

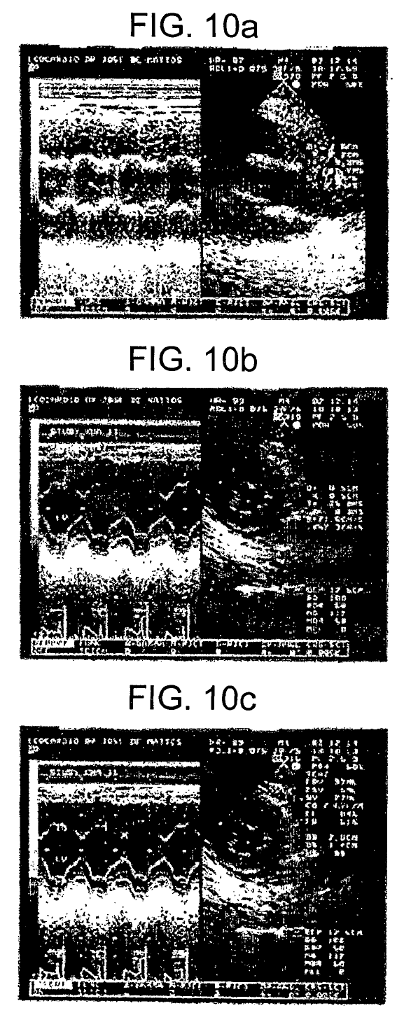

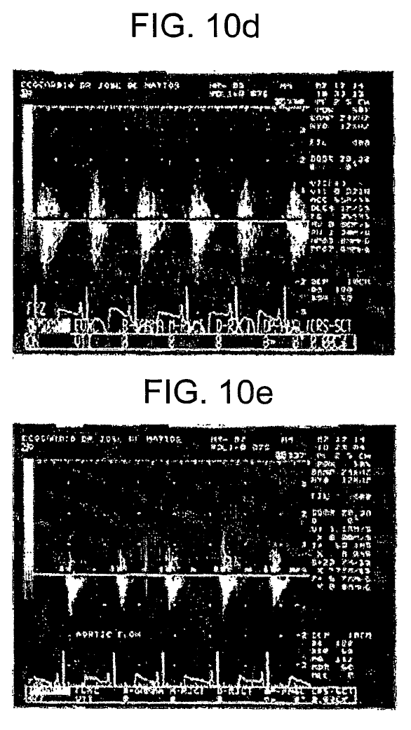

Study ID KHA 11: A 6-month-old, 28 kg neutered male sheep (ear tag number 857BR) underwent stentless aortic valve replacement. A bioprosthesis device (size 19 mm) was implanted with no complications. The surgical procedure and anesthesia recovery was uneventful and throughout the long-term holding period, the animal remained healthy and blood sampling was obtained per protocol. The animal was found dead on the 134^{th} post-operative day. FIGS. 10a to 10e show the results of the echocardiography analysis for this study. The aortic valve shows normal Echo aspects with no evidence of anatomical or functional alteration.

Although the foregoing description of the present invention has been shown and described with reference to particular embodiments and applications thereof, it has been presented for purposes of illustration and description and is not intended to be exhaustive or to limit the invention to the particular embodiments and applications disclosed. It will be apparent to those having ordinary skill in the art that a number of changes, modifications, variations, or alterations to the invention as described herein may be made, none of which depart from the spirit or scope of the present invention. The particular embodiments and applications were chosen and described to provide the best illustration of the principles of the invention and its practical application to thereby enable one of ordinary skill in the art to utilize the invention in various embodiments and with various modifications as are suited to the particular use contemplated. All such changes, modifications, variations, and alterations should therefore be seen as being within the scope of the present invention as determined by the appended claims when interpreted in accordance with the breadth to which they are fairly, legally, and equitably entitled.

1. A heart valve prosthesis having a plurality of leaflets encircling a flow opening and of size to coapt to form a valve, each leaflet having a free outflow edge at the outflow end of the leaflet, wherein the free outflow edge forms a convex (relative to the leaflet) curve in the plane of the leaflet.

2. A method for forming a heart valve prosthesis comprising the step of forming a plurality of leaflets joined to encircle a flow passage and of a size to coapt to form a valve, wherein each leaflet has a free outflow edge at the outflow end of the leaflet, wherein the free outflow edge forms a convex (relative to the leaflet) curve in the plane of the leaflet.

3. A method for forming a heart valve prosthesis comprising the steps of:
assembling the valve, by steps comprising forming a plurality of leaflets joined to encircle a flow passage and of a size to coapt to form a valve, and forming an outer sheet joined to the leaflets around an inflow end and along commissures formed where adjacent leaflets join;
after assembly of at least the leaflets and outer sheet of the valve, shaping the leaflets and/or outer sheet to a desired shape; and
fixing the leaflets and/or outer sheet of the valve in the desired shape.

4. The method of claim 3 wherein the leaflets and outer sheet are shaped to a desired shape and fixed in the desired shape, and wherein the shaping of the outer sheet is of a portion of the outer sheet on the outflow side of the join between the outer sheet and the leaflets around the inflow end.

5. A method for forming a stentless heart valve prosthesis comprising the steps of:
forming a plurality of leaflets joined to encircle a flow passage and of a size to coapt to form a valve; and
forming an outer sheet joined to the leaflets around an inflow end and along commissures formed where adjacent leaflets join;
wherein the join between the outer sheet and the leaflets around the inflow end is at the periphery of the leaflets, and the outer sheet extends by a distance between 0.3 and 4 mm beyond the join with the leaflets at the inflow end, on the inflow side of the join, or the join between the outer sheet and the leaflets around the inflow end is at the periphery of the outer sheet, and the leaflets extend by a distance between 0.3 and 4 mm beyond the join with the outer sheet at the inflow end, on the inflow side of the join.

6. The method of claim 5, further comprising the step of after assembly of at least the leaflets and outer sheet of the valve, shaping the leaflets and/or outer sheet to a desired shape and fixing the leaflets and/or outer sheet of the valve in the desired shape.

7. The method of claim 5 wherein each leaflet has a free outflow edge at the outflow end of the leaflet, wherein the free outflow edge forms a convex (relative to the leaflet) curve in the plane of the leaflet.

8. The method of claim 7 wherein the leaflets and/or outer sheet are shaped to a desired shape by inserting a shaping device into a pocket formed by a leaflet and the outer sheet.

9. The method of claim 8 wherein the shaping device is a ball formed of a resilient material, preferably cotton wool.

10. The method of claim 6 wherein the leaflets and outer sheet are assembled from pericardium which has not been fixed, and fixing the leaflets and/or outer sheet of the valve in the desired shape is performed by treatment with glutaraldehyde.

11. The method of claim 6 wherein after assembly and fixing of the valve the outer sheet is trimmed close to the join between the outer sheet and the leaflets on the outflow side of the join.

12. A valve obtainable by the method of claim 5.

13. A stentless heart valve prosthesis comprising a plurality of leaflets joined to encircle a flow passage and of a size to coapt to form a valve, an outer sheet joined to the leaflets around an inflow end and along commissures formed where adjacent leaflets join, wherein the join between the outer sheet and the leaflets around the inflow end is at the periphery of the leaflets, and the outer sheet extends by a distance between 0.3 and 4 mm beyond the join with the leaflets at the inflow end, on the inflow side of the join, or the join between the outer sheet and the leaflets around the inflow end is at the periphery of the outer sheet, and the leaflets extend by a distance between 0.3 and 4 mm beyond the join with the outer sheet at the inflow end, on the inflow side of the join.

14. The heart valve prosthesis of claim 13 wherein the outer sheet of the valve has a shape resembling the shape of natural aortic sinuses.

15. The heart valve prosthesis of claim 13 wherein the outer sheet is trimmed close to the join between the outer sheet and the leaflets on the outflow side of the join.

16. The heart valve prosthesis of claim 13 wherein each leaflet has a free outflow edge at the outflow end of the leaflet, wherein the free outflow edge forms a convex (relative to the leaflet) curve in the plane of the leaflet.

17. The heart valve prosthesis of claim 13 wherein the valve has three leaflets.

18. The heart valve prosthesis of claim 13 wherein the valve is stentless.

19. The heart valve prosthesis of claim 1 wherein the valve comprises an outer sheet joined to the leaflets around an inflow end and along commissures formed where adjacent leaflets join.

20. The heart valve prosthesis or claim 13 wherein the leaflets and/or outer sheet (where present) are formed from material other than natural valve material.

21. The heart valve prosthesis of claim 20 wherein the leaflets are formed from a sheet material.

22. The heart valve prosthesis or claim 20 wherein the leaflets and/or outer sheet are formed from a biological material.

23. The heart valve prosthesis or claim 22 wherein the leaflets and/or outer sheet are formed from pericardium.

24. The heart valve prosthesis or claim 13 wherein the leaflets are formed by a single piece of sheet material.

25. (canceled)

26. (canceled)

27. A method of repairing a heart valve comprising the step of providing a heart valve prosthesis as defined in claim 13, and suturing the heart valve prosthesis to the heart or blood vessel tissue of the patient.

28. A method of repairing a heart valve comprising the steps of:
(1) providing a valve prosthesis as defined in any one of the preceding claims, wherein the valve has an outer sheet;
(2) if not already done, trimming the outer sheet close to the join between the outer sheet and the leaflets on the outflow side of the join; and
(3) suturing the valve prosthesis to the heart or blood vessel tissue of the patient with a single suture row.

29. A stentless heart valve prosthesis suitable for replacement of the aortic root comprising an outer wall and a plurality of leaflets positioned inside the outer wall, encircling a flow opening and of size to coapt to form a valve, wherein the outer wall and leaflets are formed from material other than natural valve material.

30. A method for forming a stentless heart valve prosthesis suitable for replacement of the aortic root comprising the steps of forming an outer wall and a plurality of leaflets positioned inside the outer wall, encircling a flow opening and of size to coapt to form a valve, wherein the outer wall and leaflets are formed from material other than natural valve material.

31. A stentless heart valve prosthesis suitable for replacement of the aortic root comprising a plurality of leaflets joined to encircle a flow passage and of a size to coapt to form a valve, an outer wall joined to the leaflets around an inflow end and along commissures formed where adjacent leaflets join, wherein the outer wall and leaflets are formed from material other than natural valve material, wherein the join between the outer wall and the leaflets around the inflow end is at the periphery of the leaflets, and the outer wall extends by a distance between 0.3 and 4 mm beyond the join with the leaflets at the inflow end, on the inflow side of the join, or the join between the outer sheet and the leaflets around the inflow end is at the periphery of the outer sheet, and the leaflets extend by a distance between 0.3 and 4 mm beyond the join with the outer wall at the inflow end, on the inflow side of the join.

32. The heart valve prosthesis of claim 31 wherein the outer wall is formed from a biological material (other than natural valve material) and a non-biological, biocompatible material.

33. The heart valve prosthesis claim 32 wherein the outer wall is formed from pericardium and a woven fabric, preferably polyester (PET).

34. The heart valve prosthesis of claim 31 wherein the leaflets are formed from a biological material, for example pericardium.

35. The heart valve prosthesis of claim 31 wherein each leaflet has a free outflow edge at the outflow end of the leaflet, wherein the free outflow edge forms a convex (relative to the leaflet) curve in the plane of the leaflet.

36. The method of claim 30 comprising the steps of:
assembling the valve, by steps comprising forming a plurality of leaflets joined to encircle a flow passage and of a size to coapt to form a valve, and forming an outer wall joined to the leaflets around an inflow end and along commissures formed where adjacent leaflets join;
after assembly of at least the leaflets and outer wall of the valve, shaping the leaflets and/or outer wall to a desired shape; and
fixing the leaflets and/or outer wall of the valve in the desired shape.

37. The method of claim 30 comprising the steps of:
forming a plurality of leaflets joined to encircle a flow passage and of a size to coapt to form a valve; and
forming an outer wall joined to the leaflets around an inflow end and along commissures formed where adjacent leaflets join;
wherein the join between the outer wall and the leaflets around the inflow end is at the periphery of the leaflets, and the outer wall extends by a distance between 0.3 and 4 mm beyond the join with the leaflets at the inflow end, on the inflow side of the join; or the join between the outer wall and the leaflets around the inflow end is at the periphery of the outer wall, and the leaflet extends by a distance between 0.3 and 4 mm beyond the join with the outer wall at the inflow end, on the inflow side of the join.

38. The method of claim 37 further comprising the steps of after assembly of at least the leaflets and outer wall of the valve, shaping the leaflets and/or outer wall to a desired shape and fixing the leaflets and/or outer wall of the valve in the desired shape.

39. The method of claim 38 wherein the leaflets and/or outer sheet are shaped by inserting a shaping device into a pocket formed by a leaflet and the outer wall.

40. The method of claim 39 wherein the shaping device is a ball formed of a resilient material, for example cotton wool.

41. The method of claim 38 wherein the outer wall is shaped to a desired shape and fixed in the desired shape.

42. The method of claim 38 wherein the leaflets and outer sheet are assembled from pericardium which has not been fully fixed, and fixing the leaflets and/or outer sheet of the valve in the desired shape is performed by treatment with glutaraldehyde.

43. The method of claim 37 wherein the outer wall or sheet has a height (ho) that is between 1 and 15 cm, preferably between 4 and 12 cm, still more preferably between about 8 and 10 cm greater than the maximum height (h) of the leaflets.

44. A stentless heart valve prosthesis suitable for replacement of the aortic root obtained or obtainable by a method according to claim 37.

45. The heart valve prosthesis of claim 44 wherein the outer sheet of the valve has a shape resembling the shape of natural aortic sinuses.

46. The heart valve prosthesis of claim 44 wherein the valve has three leaflets.

47. The heart valve prosthesis of claim 44 wherein the leaflets are formed by a single piece of sheet material.

48. (canceled)

49. (canceled)

50. A method for repairing a heart valve comprising the step of providing a valve prosthesis according to claim 44, and suturing the valve prosthesis to the heart or blood vessel tissue of the patient.

51. The method of claim 50 comprising the step of trimming the outer wall to the desired length, depending on the extent of aortic tissue to be replaced.
